(19) United States
(12) Patent Application Publication
(10) Pub. No.: US 2018/0192895 A1
(43) Pub. Date: Jul. 12, 2018

(54) DETECTION OF PERIPHERAL ARTERIAL DISEASE

(71) Applicant: Yissum Research Development Company of the Hebrew University of Jerusalem Ltd., Jerusalem (IL)

(72) Inventors: Ram WEISS, Haifa (IL); Ariel SHOCHAT, Jerusalem (IL); Chanan VINITZKY, Petach-Tikva (IL); Yaacov LACHER, Jerusalem (IL); Amnon BUXBOIM, Tel-Aviv (IL); Yaakov NAHMIAS, Mevaserer Zion (IL)

(21) Appl. No.: 15/738,588

(22) PCT Filed: Jun. 21, 2016
(86) PCT No.: PCT/IL2016/050657

§ 371 Date: Dec. 21, 2017

Related U.S. Application Data
(60) Provisional application No. 62/182,587, filed on Jun. 21, 2015.

(51) Int. Cl. A61B 5/02; A61B 5/022; A61B 5/026; A61B 5/024

(52) U.S. Cl. CPC A61B 5/02007; A61B 5/022; A61B 5/01; A61B 5/024; A61B 5/026

(57) ABSTRACT

A system for screening for Peripheral Artery Disease (PAD), including a plurality of instruments for measuring presence of blood flow derived physiological parameters, a data collection unit attached to each one of the instruments for receiving signals from each one of the instruments, and a data analysis unit for determining a likelihood of presence of PAD based, at least in part, on the received signals. Related apparatus and methods are also described.

### RELATED APPLICATION/S

This application is a PCT Application claiming priority of U.S. Provisional Patent Application No. 62/182,587 filed 21 Jun. 2015.

The contents of all of the above applications are incorporated by reference as if fully set forth herein.

### FIELD AND BACKGROUND OF THE INVENTION

The present invention, in some embodiments thereof, relates to a system and methods for assessing limb circulation and detecting Peripheral Arterial Disease (PAD), and, more particularly, but not exclusively, to a system and methods for detecting a possible presence and location of partial or advanced arterial occlusion.

Peripheral artery disease (PAD), also known as peripheral vascular disease is a narrowing of the arteries other than those that supply the heart or the brain.

Peripheral artery disease commonly affects the legs, but other arteries may also be involved. A classic symptom is leg pain when walking which resolves with rest. Other symptoms including skin ulcers, bluish skin, cold skin, or poor nail and hair growth may occur in the affected leg. Complications may include an infection or tissue necrosis which may require amputation; coronary artery disease, or stroke. In up to half of people there are no symptoms despite the presence of substantial and clinically relevant lower limb PAD.

PAD is typically diagnosed by a physician measuring blood pressure on an arm of a patient and then measuring blood pressure on an ankle, and finding an ankle-brachial index less than 0.90, which is the systolic blood pressure below the ankle divided by the systolic blood pressure of the arm.

Duplex ultrasonography and angiography may also be used. Angiography is more precise and allows for accurate planning and performance of invasive treatment; however, it is associated with greater risks.

Additional background art includes:

An article titled “A Study on Visualization of Auscultation-based Blood Pressure Measurement” by Katsuki et al., published in SASIMI 2015 Proceedings.

An article titled “Patient-Friendly Detection of Early Peripheral Arterial Diseases (PAD) by Budgeted Sensor Selection” by Wang et al., published in PervasiveHealth 2012, May 21-24, San Diego, United States, DOI 10.4108/icst.pervasivehealth.2012.249068.

The disclosures of all references mentioned above and throughout the present specification, as well as the disclosures of all references mentioned in those references, are hereby incorporated herein by reference.

### SUMMARY OF THE INVENTION

The present invention, in some embodiments thereof, relates to a system and methods for assessing limb circulation and screening for and detection of Peripheral Arterial Disease (PAD), and, more particularly, but not exclusively, to a system and methods for detecting a possible presence and location of an partial or advanced arterial occlusion.

An aspect of some embodiments of the invention relates to measuring indications of blood flow, blood properties and tissue properties and its effects at the same time at different locations along a patient's limb. Such measurement of physiological parameters associated with blood flow measurement under same conditions rather than spaced apart in time is a potentially more accurate detector of PAD. Such measurement of blood flow measurement under same conditions at different locations potentially enables detecting an arterial occlusion based on blood flow measurement differences the different locations.

An aspect of some embodiments of the invention relates to measuring relative changes between multiple sensors on a same limb and/or between limbs, and/or dynamics of the changes following post obstructive flow, optionally within a location on a limb and/or between proximal and distal locations on a limb. The changes are optionally evaluated over time and/or over positional changes of an affected limb in comparison to another limb.

According to an aspect of some embodiments of the present invention there is provided a system for screening for Peripheral Artery Disease (PAD), including a plurality of instruments for measuring presence of blood flow derived physiological parameters, a data collection unit attached to each one of the instruments for receiving signals from each one of the instruments, and a data analysis unit for determining a likelihood of presence of PAD based, at least in part, on the received signals.

According to some embodiments of the invention, the plurality of instruments for measuring presence of blood flow derived physiological parameters are configured to dynamically measure the parameters over time.

According to some embodiments of the invention, the plurality of instruments for measuring presence of blood flow derived physiological parameters are configured to dynamically measure the parameters in response to positional maneuvers of the limbs.

According to some embodiments of the invention, the data analysis unit is arranged to determine a likelihood of presence of PAD based, at least in part, additionally on anthropometric, demographic and clinical data.

According to some embodiments of the invention, further including a plurality of inflatable and deflatable cuffs for temporarily obstructing the circulation in a limb.

According to some embodiments of the invention, further including an electric pump for inflating the inflatable cuffs and an inflation controller for controlling inflation and release of pressure of the inflatable cuffs.

According to some embodiments of the invention, further including the inflation controller arranged to maintain same pressure at each one of the plurality of inflatable cuffs.

According to some embodiments of the invention, further including an adjustable stirrup for maintaining a limb of a patient at a specific angle.

According to some embodiments of the invention, further including an adjustable stirrup for maintaining a limb of a patient at a specific position and changing the position in a pre-programmed manner.

According to some embodiments of the invention, further including an adjustable thigh stirrup for supporting a thigh of a patient and an adjustable leg stirrup for supporting a leg of a patient.

According to some embodiments of the invention, further including the adjustable thigh stirrup and the adjustable leg stirrup functionally attached to a motor for adjusting an angle between the thigh of the patient and the leg of the patient.

According to some embodiments of the invention, further including an angle measurement device for measuring the angle between the thigh stirrup and the leg stirrup and transmitting the angle measurement.

According to some embodiments of the invention, further including a sleeve for placing at least one of the plurality of the instruments at a location behind a knee of a patient.

According to some embodiments of the invention, further including a sleeve for placing at least one of the plurality of the instruments at a medial or lateral aspect of an ankle of a patient.

According to some embodiments of the invention, further including a sleeve for placing at least one of the plurality of the instruments at a toe of a patient.

According to some embodiments of the invention, further including a sleeve for placing at least one of the plurality of the instruments in a vicinity of a dorsalis pedis artery.

According to some embodiments of the invention, further including a sleeve for placing at least one of the plurality of the instruments at an inner elbow of a patient.

According to some embodiments of the invention, at least one of the instruments includes a piezoelectric pressure sensor.

According to some embodiments of the invention, at least one of the instruments includes one or more of a membrane pressure sensor; a capacitor pressure sensor; a microphone; and a pressure sensor of some other type.

According to some embodiments of the invention, at least one of the instruments includes a pulse oxymetry sensor.

According to some embodiments of the invention, at least one of the instruments includes a thermistor based temperature sensor.

According to an aspect of some embodiments of the present invention there is provided a method for determining a likelihood of presence of Peripheral Artery Disease (PAD), including attaching a first instrument for detecting presence of blood flow derived physiological parameters to an arm of a patient, attaching a second instrument for detecting presence of blood flow derived physiological parameters to a leg of a patient, obstructing blood flow in the arm and the leg followed by relieving the obstructing, collecting signals from the first instrument and the second instrument, and analyzing the signals to determine a likelihood of presence of PAD, thereby determining a likelihood of presence of PAD.

According to some embodiments of the invention, further including changing a limb angle, collecting signals from the first instrument and the second instrument at a changed limb angle, and analyzing the signals to determine a likelihood of presence of PAD, based, at least in part, on comparing the signals at different angles.

According to some embodiments of the invention, the first instrument and the second instrument are configured to dynamically measure the parameters over time.

According to some embodiments of the invention, the analyzing the signals further includes determining the likelihood of presence of PAD based, at least in part, additionally on anthropometric, demographic and clinical data.

According to some embodiments of the invention, further including using a determination of the likelihood of presence of PAD to drive a clinical decision associated with the determination.

According to some embodiments of the invention, further including using a determination of the likelihood of presence of PAD to drive a clinical intervention associated with the determination.

According to some embodiments of the invention, the second instrument is attached behind a knee of the patient.

According to some embodiments of the invention, the obstructing blood flow in the arm and the leg includes obstructing the blood flow until signals from the first instrument and the second instrument indicate no blood is flowing, followed by releasing the obstruction.

According to some embodiments of the invention, further including determining a location of an arterial occlusion based, at least in part, on the analyzing the signals.

According to some embodiments of the invention, further including determining a location of a partial arterial occlusion based, at least in part, on the analyzing the signals.

According to some embodiments of the invention, the obstructing blood flow includes controlling a time of inflating an arm cuff and a thigh cuff.

According to some embodiments of the invention, the obstructing blood flow includes simultaneously inflating an arm cuff and a thigh cuff.

According to some embodiments of the invention, the obstructing blood flow includes maintaining a same pressure at each one of a plurality of inflatable cuffs.

According to some embodiments of the invention, further including placing a leg of a patient in an adjustable stirrup and measuring an angle between the leg and a thigh of the patient.

According to some embodiments of the invention, further including placing a thigh of a patient in an adjustable thigh stirrup and placing a leg of the patient in an adjustable leg stirrup and measuring an angle between the leg stirrup and the thigh stirrup.

According to some embodiments of the invention, further including using a motor to automatically adjust the angle between the leg stirrup and the thigh stirrup.

According to some embodiments of the invention, further including using a motor to automatically adjust a thigh angle between a leg and a torso of a supine patient.

According to some embodiments of the invention, further including using a controller to instruct the motor to automatically adjust the angle between the leg stirrup and the thigh stirrup to a plurality of different angles, and instruct a data collection unit to collect signals from at least the first instrument and the second instrument for each one of the plurality of different angles.

According to some embodiments of the invention, analyzing the signals to determine a likelihood of presence of PAD includes analyzing the signals collected for each one of the plurality of different angles and an angle measured between the leg stirrup and the thigh stirrup.

According to some embodiments of the invention, a plurality of instruments is attached to a leg of a patient for detecting presence of blood flow.

According to some embodiments of the invention, further including determining a location of an arterial occlusion based, at least in part, on detecting a difference in blood flow and/or flow derived signals between the plurality of instruments.

According to some embodiments of the invention, further including determining a location of a partial arterial occlusion based, at least in part, on detecting a difference in blood flow between the plurality of instruments.

According to some embodiments of the invention, at least one of the plurality of instruments is attached behind a knee of the patient.

According to some embodiments of the invention, the at least one of the plurality of instruments which is attached behind the knee of the patient is attached using a sleeve arranged for locating the instrument behind the knee.

According to some embodiments of the invention, at least one of the plurality of instruments is attached behind an ankle of the patient.

According to some embodiments of the invention, the at least one of the plurality of instruments which is attached behind the ankle of the patient is attached using a sleeve arranged for locating the instrument behind the ankle.

According to some embodiments of the invention, at least one of the plurality of instruments is attached to a toe of the patient.

According to some embodiments of the invention, the at least one of the plurality of instruments which is attached to the toe of the patient is attached using a sleeve arranged for attaching to the toe.

According to some embodiments of the invention, the at least one of the plurality of instruments is attached using a sleeve.

According to an aspect of some embodiments of the present invention there is provided a method for determining a likelihood of presence of Peripheral Artery Disease (PAD), including attaching a first instrument for detecting presence of blood flow to a proximal portion of a limb of a patient, attaching a second instrument for detecting presence of blood flow to a distal portion of the limb of the patient, obstructing blood flow in the upper portion, collecting initial signals from the first instrument and the second instrument, changing an angle between the proximal portion and the distal portion, obstructing blood flow in the upper portion, collecting additional signals from the first instrument and the second instrument, and comparing the initial signals to the additional signals to determine a likelihood of presence of PAD, thereby determining a likelihood of presence of PAD.

According to some embodiments of the invention, the proximal portion is a thigh and the distal portion is a leg.

According to some embodiments of the invention, the collecting initial signals and additional signals from the first instrument and the second instrument includes collecting a plurality of signals from a plurality of sensors in at least one of the first instrument and the second instrument, and comparing the plurality of signals from a plurality of sensors and discarding at least one signal from the plurality of signals from the plurality of sensors.

According to some embodiments of the invention, the obstructing blood flow in the upper portion includes obstructing the blood flow until signals from the first instrument and the second instrument indicate no blood is flowing, followed by releasing the obstruction.

According to some embodiments of the invention, further including repeating the changing the angle between the upper portion and the lower portion and the collecting additional signals from the first instrument and the second instrument a plurality of times.

According to some embodiments of the invention, the changing the angle between the upper portion and the lower portion by an increment of a specific angle.

According to some embodiments of the invention, the specific angle is one selected from a group consisting of ten degrees, twenty degrees, thirty degrees, forty five degrees, sixty degrees, eighty degrees, and ninety degrees.

According to some embodiments of the invention, the changing the angle between the upper portion and the lower portion includes continuing the changing the angle from a straightest angle to a most-bent angle.

According to some embodiments of the invention, the changing the angle between the upper portion and the lower portion comprises continuous changing of the angle between two predetermined angles.

According to some embodiments of the invention, the changing the angle between the upper portion and the lower portion includes stopping the changing the angle when one of the first instrument and the second instrument indicate a drop in blood flow by a specific percentage.

According to some embodiments of the invention, the changing the angle between the upper portion and the lower portion includes stopping the changing the angle when one of the first instrument and the second instrument indicate no blood is flowing.

According to an aspect of some embodiments of the present invention there is provided a method for determining a presence and location of an arterial occlusion, including attaching a first instrument for detecting presence of blood flow to a proximal portion of a limb of a patient, attaching a second instrument for detecting presence of blood flow to a distal of the limb of the patient, obstructing blood from flowing in the upper portion, releasing the obstruction, collecting signals from the first instrument and the second instrument, and analyzing the signals to determine a location of an arterial occlusion, thereby determining a location of an arterial occlusion.

According to some embodiments of the invention, further including attaching a third instrument for detecting presence of blood flow to another limb of the patient, obstructing blood from flowing in the other limb, releasing the obstruction in the other limb, collecting signals from the third instrument, and analyzing the signals to determine a location of an arterial occlusion.

According to some embodiments of the invention, the determining the location of the arterial occlusion is performed by comparing blood flow in the first instrument and in the second instrument to blood flow in the third instrument, based on their anatomical location and/or distance from the hip.

According to some embodiments of the invention, when the analyzing determines that the first instrument measured blood flow higher than the second instrument by a specific threshold, the location of the arterial occlusion is determined to be between the first instrument and the second instrument.

According to some embodiments of the invention, when the analyzing determines that both the first instrument and the second instrument measure a decrease in blood flow relative to the third instrument the location of the arterial occlusion is determined to be upstream of the first instrument.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a simplified block diagram illustration of an example embodiment of the invention;

FIG. 1B is a simplified block diagram illustration of an example embodiment of the invention;

FIG. 1C is a simplified block diagram illustration of an example embodiment of the invention;

FIG. 2A is a simplified flow chart illustration of an example embodiment of the invention;

FIG. 2B is a simplified flow chart illustration of an example embodiment of the invention;

FIG. 2C is a simplified flow chart illustration of an example embodiment of the invention;

FIG. 2D is a simplified flow chart illustration of an example embodiment of the invention;

FIG. 3A is a simplified illustration of an example embodiment of the invention;

FIG. 3B is a simplified illustration of an example embodiment of the invention;

FIG. 4 is a simplified illustration of a piezoelectric crystal for use as a piezoelectric sensor according to an example embodiment of the invention; and

FIG. 5 is a simplified illustration of an array of sensors according to an example embodiment of the invention.

### DESCRIPTION OF SPECIFIC EMBODIMENTS OF THE INVENTION

The present invention, in some embodiments thereof, relates to a system and methods for detecting Peripheral Arterial Disease (PAD), and, more particularly, but not exclusively, to a system and methods for detecting a possible presence and location of an partial or advanced arterial occlusion.

An aspect of some embodiments of the invention relates to measuring indications of physiological parameters affected by blood flow at the same time at different locations along a patient's limb. Such measurements of physiological parameters affected by blood flow under same conditions rather than spaced apart in time is a potentially a more accurate detector of PAD. Such measurement of physiological parameters affected by blood flow measurement under same conditions at different locations potentially enables detecting an arterial occlusion based on blood flow measurement differences the different locations.

An aspect of some embodiments of the invention relates to measuring relative changes between multiple sensors on a same limb and/or between limbs, and/or dynamics of the changes following post obstructive flow, optionally within a location on a limb and/or between proximal and distal locations on a limb. The changes are optionally evaluated over time and positional changes.

### Introduction

Screening for PAD may be relatively simple. A physician may use a widely available blood pressure device for measuring blood pressure on an arm of a patient and then measuring blood pressure on an ankle, or vice versa, and calculate an ankle-brachial index (ABI) of less than 0.90. A measurement of a blood pressure difference between the arm and the ankle is typically taken to imply a problem in blood flow at the limb showing depressed blood pressure.

However, the simple attempt at diagnosis suffers from several weaknesses:

blood pressure of a single patient can vary over a very short time period, so that a significant error may be introduced when calculating the ABI;

blood pressure measurements depend on the position of a patient, which is typically achieved by performing the simple measurement in one position only, such as lying down. Such a position may not expose symptoms of PAD, which may be exposed when, for example, a leg or a thigh is bent relative to a torso of a patient.

Subsequent blood pressure measurements of a single patient can vary if performed by a human operator of a blood pressure device, so that an error is introduced when calculating the ABI.

More accurate blood flow measurement methods like the above-mentioned Duplex ultrasonography and angiography are much more expensive, require specially trained personnel, and are typically not suitable for screening patients for PAD. They are typically used when a physician indicates that a patient is highly likely to be suffering from PAD based on an initial blood pressure test as described above.

### Overview

An aspect of some embodiments of the invention relates to instrumental measurement of physiological parameters affected by blood flow at a same time at different locations at varying conditions, so as to minimize differences over time.

In some embodiments, when cuffs are inflated in order to block blood flow at the different locations, the cuffs are inflated by one source for pressurized air, thereby producing a same pressure at the different locations at the same time. The deflation is optionally simultaneous as well, in which case post obstructive flow is optionally measured at all limbs at a same time, in parallel.

An aspect of some embodiments of the invention relates to instrumental measurement of parameters affected by blood flow so as to minimize human errors introduced into measurement.

An aspect of some embodiments of the invention relates to instrumental measurement of blood flow so as to minimize human errors introduced into measurement.

An aspect of some embodiments of the invention relates to collecting measurements from a repeatable instrument, which minimizes error. Obtaining repeated measurements by a same sensor/location/technique combination potentially enables determining more accurate thresholds for assessing high, medium or low likelihood of PAD.

Parameters which are measured optionally include one or more, yet are not limited to, blood pressure and/or oxygen saturation and/or skin temperature and their time and position dependent dynamics in response post blood flow obstruction and leg position changes. For example, differences in the recovery time of oxygen saturation distal to the obstruction between patent and partially obstructed leg arteries ranges from 1-2 seconds. Another example is a change in blood flow in response to varying leg angles which may be minor in patent arteries yet are potentially substantial in partially obliterated arteries, showing, by way of a non-limiting example, 20-30% difference in flow upon a 90 degree position change. Another example is skin temperature distal compared to proximal of an obstruction which can vary by 1-1.5 degrees Celsius.

In some embodiments, a difference of greater than a specific percentage in blood flow related parameters between instruments on a same limb of a patient, and/or between different limbs of a patient, are considered as indicating a high likelihood of PAD.

An aspect of some embodiments of the invention relates to collecting data for patients as measured by a system as described herein, and PAD diagnoses made for the patients and determining specific thresholds for the system for differentiating between high likelihood of PAD, medium likelihood of PAD, low likelihood of PAD, and optionally no likelihood of PAD.

### Anatomic Locations for Instrumentation

An aspect of some embodiments of the invention relates to locations of instrumentation, that is, locations of placing measuring sensors for detecting signals indicating physiological parameters affected by blood flow and/or pulse.

In some embodiments, two or more locations from the list below are selected for locating sensors:

arm;

behind the knee (popliteal fossa);

ankle (vicinity of tibilais posterior, medial aspect);

toe(s); and

dorsalis pedis artery region.

### Detecting a Location of an Arterial Occlusion

An aspect of some embodiments of the invention relates to determining a location of an arterial occlusion based on detecting a relative and/or absolute difference in physiological parameters affected by blood flow between instruments located at different locations along a limb, proximal and distal to the occlusion. Detecting these differences potentially enables to deduce that an obstruction exists downstream of an instrument showing a relatively greater or different response to high blood flow, post obstructive, in comparison to a distal instrument showing relatively lower response of parameters affected by the lower blood flow. The differences in responses of the multiple parameters tested are not all necessarily in direct relation, some may be in inverse relation, that is, some parameters may increase while other parameters may decrease distal to an arterial obstruction while others may have a different dynamic pattern of change in time and position.

In some embodiments measurement of physiological parameters affected by blood flow and detection of presence of an occlusion are optionally performed at specific torso-thigh, thigh-leg, or leg-to-leg relative angles.

In some embodiments, when presence of an occlusion is suspected, additional measurements are optionally made at additional angles, which potentially provides data regarding angles at which the occlusion affects physiological parameters affected by blood flow and potentially other angles at which the occlusion affects the physiological parameters affected by blood flow more, or less, or not at all.

In some embodiments, when presence of an occlusion in a leg is suspected, additional measurements are optionally made to the other leg, optionally at various angles as described above.

### Attaching Sensors to a Patient at Specific Anatomical Locations

An aspect of some embodiments of the invention relates to patches/sleeves for placement of instrumentation at the above locations. The sleeves are optionally differently designed specifically for the different locations, so as to enable a care-giver to place instrumentation at a correct location on a patient's body, and at a same location time after time in a repeatable fashion, optionally without need for special training.

By way of a non-limiting example an arm sleeve is optionally similar to current elbow support, with a location for arm sensor(s) marked on an inner-elbow of the arm sleeve and/or a pocket designed in the arm sleeve for locating the sensor(s).

By way of a non-limiting example a knee sleeve is optionally similar to current knee support, with a location for behind the knee sensor(s) marked on the knee sleeve and/or a pocket designed in the knee sleeve for locating the sensor(s).

By way of a non-limiting example an ankle sleeve is optionally similar to current ankle support, with a location for a medial aspect of the ankle sensor(s) optionally marked on the ankle sleeve and/or a pocket designed in the ankle sleeve for locating the sensor(s).

By way of a non-limiting example a toe sleeve is optionally similar to current toe or finger bandages, with a location for sensor(s) marked on the toe sleeve and/or a pocket designed in the toe sleeve for locating the sensor(s).

In some embodiments, two or more locations from the list below are optionally selected for placing cuffs for blood flow obstruction:

arm; and

distal thigh, above the knee.

### Sensors

Various sensors are described below. In various embodiments, different combinations of sensors are used. Optionally one or more types of sensor are optionally used at an anatomic location.

In some embodiments the sensor types optionally include multi sensor instrumentation at multiple measurement points optionally including microphones, piezo-electric elements, thermometers, thermistors, thermocouples, pyroelectric, optical heart rate sensors, membranes, and tissue oxygen saturation sensors and impedance.

### Piezoelectric Sensor(s)

In some embodiments a pulse sensor is attached to a patient at any one of the above-mentioned locations, to determine whether a pulse is felt, indicating a flow of blood.

In some embodiments the pulse sensor uses one or more piezoelectric pressure sensors.

In some embodiments the pulse sensor uses one or more piezoelectric pressure sensors which can detect pressures down to 1 microbar.

In some embodiments a single piezoelectric sensor is used at an anatomical location, and provides an electric signal corresponding to pressure. Analyzing the electric signal potentially enables detecting when a pulse is present and when the pulse is absent.

In some embodiments the piezoelectric sensor reacts to pressure produced by pulse including measuring a shape of a pressure signal produced by the pulse.

In some embodiments more than one piezoelectric sensors are used, and each provides an electric signal corresponding to pressure. Analyzing the electric signal from each one of the sensors potentially enables detecting when a pulse is present and when the pulse is absent from that sensor. In some embodiments detecting a pulse by even one of the sensors is enough to determine that a pulse exists at the anatomical location at which the sensors are attached. In such embodiments using the several sensors enables measuring pulse even if the sensors are not placed accurately, since several sensors cover a larger area than one, and several are likely to detect a pulse even when placed inaccurately.

### Optical Sensor(s)

In some embodiments a pulse oximetry sensor is attached to a patient at any one of the above-mentioned locations, to determine whether arterial blood arrives at the location, indicating a flow of arterial blood.

In some embodiments the pulse oximetry sensor used is a reflectance sensor.

In some embodiments the pulse oximetry sensor includes one or more light emitters, such as, by way of a non-limiting example, a Light Emitting Diode (LED), and a light sensor, such as, by way of a non-limiting example, a photodiode.

In some embodiments, at least at some of the anatomical locations for thin sections of a body such as a toe, a finger, or an ankle of a young patient, the pulse oximetry sensor used is a transmission sensor.

In some embodiments one or more pulse oximetry sensors are used at a single anatomical location.

In some embodiments a single pulse oximetry sensor is used, and provides a signal corresponding to presence of arterial blood flow. Analyzing the signal potentially enables detecting when arterial blood flow is present and when the arterial blood flow is absent.

In some embodiments more than one pulse oximetry sensor are used, and each provides a signal corresponding to presence of arterial blood flow. Analyzing the signal from each one of the sensors potentially enables detecting when arterial blood flow is present and when the arterial blood flow is absent from that sensor. In some embodiments detecting arterial blood flow by even one of the sensors is enough to determine that arterial blood flow exists at the anatomical location at which the sensors are attached.

In such embodiments using the several sensors enables measuring arterial blood flow even if the sensors are not placed accurately, since several sensors cover a larger area than one, and several are likely to detect arterial blood flow even when placed inaccurately.

Present pulse oxymetry sensors typically use two wavelengths, red and infrared, to measure oxygen saturation in blood. In some embodiments pulse oxymetry sensors are included which use more than two wavelengths.

### Thermistor(s)

An aspect of some embodiments of the invention relates to measuring temperature at a location on a patient's body as an indicator of blood flow.

In some embodiments a thermistor is attached to a patient at any one of the above-mentioned locations, to measure temperature at the location, indicating whether a flow of blood reaches the location.

In some embodiments one or more thermistors are used at a single anatomical location.

In some embodiments a single thermistor is used, and provides a signal corresponding to presence of blood flow. Analyzing the signal potentially enables detecting when blood flow is present and when the blood flow is absent.

In some embodiments more than one thermistor is used, and each provides a signal corresponding to presence of blood flow. Analyzing the signal from each one of the sensors potentially enables detecting when blood flow is present and when the blood flow is absent from that sensor. In some embodiments detecting blood flow by even one of the sensors is enough to determine that blood flow exists at the anatomical location at which the sensors are attached.

In such embodiments using the several sensors enables measuring blood flow even if the sensors are not placed accurately, since several sensors cover a larger area than one, and several are likely to detect blood flow even when placed inaccurately.

It is noted that the thermistor described above is intended as a non-limiting example embodiment of a temperature measurement sensor.

In some embodiments a resistance temperature detector (RTD) is optionally used for the uses described above with reference to the thermistor.

### Thermocouple

In some embodiments a thermocouple is attached to a patient at any one of the above-mentioned locations, to measure temperature at the location, indicating whether a flow of blood reaches the location.

In some embodiments one or more thermocouples are used at a single anatomical location.

In some embodiments a single thermocouple is used, and provides a signal corresponding to presence of blood flow. Analyzing the signal potentially enables detecting when blood flow is present and when the blood flow is absent.

In some embodiments more than one thermocouple is used, and each provides a signal corresponding to presence of blood flow. Analyzing the signal from each one of the sensors potentially enables detecting when blood flow is present and when the blood flow is absent from that sensor. In some embodiments detecting blood flow by even one of the sensors is enough to determine that blood flow exists at the anatomical location at which the sensors are attached.

In such embodiments using the several sensors enables measuring blood flow even if the sensors are not placed accurately, since several sensors cover a larger area than one, and several are likely to detect blood flow even when placed inaccurately.

It is noted that the thermocouple described above is intended as a non-limiting example embodiment of a temperature measurement sensor.

### Auscultation Sensor(s)

An aspect of some embodiments of the invention relates to detecting blood flow by using an auscultation sensor.

In some embodiments an auscultation sensor is attached to a patient at any one of the above-mentioned locations, to detect blood flow at the location, indicating whether a flow of blood reaches the location.

In some embodiments one or more auscultation sensor(s) are used at a single anatomical location.

In some embodiments a single auscultation sensor is used, and provides a signal corresponding to presence of blood flow. Analyzing the signal potentially enables detecting when blood flow is present and when the blood flow is absent.

In some embodiments more than one auscultation sensor is used, and each provides a signal corresponding to presence of blood flow. Analyzing the signal from each one of the sensors potentially enables detecting when blood flow is present and when the blood flow is absent from that sensor. In some embodiments detecting blood flow by even one of the sensors is enough to determine that blood flow exists at the anatomical location at which the sensors are attached.

In such embodiments using the several sensors enables measuring blood flow even if the sensors are not placed accurately, since several sensors cover a larger area than one, and several are likely to detect blood flow even when placed inaccurately.

### Simultaneous Measurement

An aspect of some embodiments of the invention relates to measuring at different location of a patient's body at a same time, thereby potentially eliminating problems which time variations of blood flow and/or blood pressure measurement.

In some embodiments, when cuffs are inflated in order to block blood flow at the different locations, the cuffs are inflated by one source for pressurized air, thereby producing a same pressure at the different locations at the same time.

In some embodiments one cuff is placed on a patient's arm and one cuff on a patient's thigh.

### Patient Position

An aspect of some embodiments of the invention relates to a patient's position and the position of the patient's limbs when measurements are taken.

In some embodiments the patient is placed upon a device which controls at what angle the patient's knee is bent relative to the thigh and/or thigh bent with straight leg raised.

In some embodiments the patient is placed upon a device which controls at what angle the patient's thigh is bent relative to the torso in the supine or sitting position.

In some embodiments the patient is placed upon a device which controls an angle of the patient's torso relative to a level, such as an inclined bed.

In some embodiments, controlling the position of a limb such as the thigh and/or the leg includes placing the limb on an adjustable platform or stirrup and optionally changing the attitude of the platform/stirrup relative to another limb and/or a platform/stirrup supporting the other limb.

In some embodiments a patient is measured using one or more of the following patient positions: sitting and lying supine.

In some embodiments, one or more leg positions of a complete range possible at the above-mentioned patient positions are measured. By way of some non-limiting examples, when lying down, angle between thigh and leg changes; and when sitting, angle changes from the knee down.

In some embodiments the patient is positioned or requested to be at a pre-set position, such as lying down supine, sitting on a chair, or at an intermediate position.

An aspect of some embodiments of the invention relates providing automatic control of the patient's position and the position of the patient's limbs when measurements are taken, and/or automatic measurement of the positions of the patient's body and limbs.

An aspect of some embodiments of the invention relates to providing control over relative torso-thigh angle and relative thigh-leg angle of a patient during blood flow measurement, which can potentially improve detection of PAD and potentially improve accuracy of medical data records.

In some embodiments measurements are performed at discrete pre-defined angles.

In some embodiments measurements are performed continuously while the angle is dynamically changed over a range of angles.

In some embodiments, the range of angles starts from a straight leg and the angle is changed until the leg is bent as far as a patient can bend it.

In some embodiments, an increment by which the leg is bent is by a specific angle, such as 5 degrees, 10 degrees, 15 degrees, 20 degrees, 30 degrees, 45 degrees and 60 degrees.

In some embodiments, an increment by which the leg is bent is by a specific angle which is likely produce a marked difference in blood flow in patients with PAD.

In some embodiments, the range of angles starts from a straight leg and the angle is changed until the leg is bent until measurements detect a likelihood of PAD.

In some embodiments the angle is recorded with the measurements.

In some embodiments a range of angles measured depends on a patient's baseline position. If the patient is lying supine, a baseline angle is 0 degrees, that is, the leg is a straight extension of the torso, and the angle between the leg and a thigh may optionally change up to 90 degrees. If the patient is sitting, the baseline angle is optionally 90 degrees, that is, the leg is optionally at 90 degrees to the thigh, and the angle may change up to 180 degrees, that is, the leg bent back until touching the thigh.

In some embodiments a continuous measurement is made of the blood flow related parameters while changing an angle. In some embodiments measurements are made at two extremes of the range of bending, and optionally at a mid-point angle. In some embodiments, by way of a non-limiting example when lying down, the following discrete angles are optionally used for measuring: 0 degrees, 30 degrees, 60 degrees 90 degrees.

### Computerized Collection of Data

An aspect of some embodiments of the invention relates to automatic collection of data produced by all the sensors used in instrumentation.

In some embodiments all the sensors are collected to an automated data gathering device and/or computer.

In some embodiments a system for automatic control of the patient's position and the position of the patient's limbs provides position data which is collected to an automated data gathering device and/or computer.

### Analysis of Data

An aspect of some embodiments of the invention relates to analysis of data collected from the measuring instruments, and/or an optional patient positioning system, and/or patient anthropometrics, demographics and clinical data entered by a caregiver, and/or such data retrieved from a medical record/database. Such data may include, by way of some non-limiting examples, anthropometric data, use of medications, smoking, height, weight, and so on.

The analysis of measurement data and/or patient medical data and/or patient demographic and/or anthropometric data is optionally performed by a pre-programmed algorithm.

In some embodiments the algorithm utilizes dynamic angle and time specific changes in the measured parameters along with additional anthropometric/clinical/demographic data to provide systemic and anatomical leg specific likelihood assessment for the presence clinically significant PAD.

In some embodiments, a system as described herein is used to measure blood flow derived physiological parameters and optionally thigh-leg and/or thigh torso angles, and optionally collect patient anthropometric and medical data, and a physician determines whether a patient has a high likelihood of PAD, a low likelihood of PAD, or requires additional testing. The above-mentioned three possible physicians decisions/diagnoses are optionally recorded. A learning algorithm optionally learns from a number of tests and physicians decisions how to transform input data into an output of a likelihood of PAD, optionally at three levels as described above for a physician's decision.

In some embodiments, a system as described herein is used to measure blood flow derived physiological parameters and optionally thigh-leg and/or thigh torso angles, and optionally collect patient anthropometric and medical data, and a physician determines where a patient is likely to have blood flow occlusion, and whether the likelihood is high, low, or requires additional testing. The above-mentioned three possible physicians decisions/diagnoses are optionally recorded. A learning algorithm optionally learns from a number of tests and physicians decisions how to transform input data into an output of a location of an occlusion, optionally at three levels as described above for a physician's decision.

In some embodiments a PAD screening device is optionally based on a system of multi-sensor heads which enables a determination of limb arterial blockage location and state. The multi-sensor heads are optionally placed along a limb in order to measure physiological vital signs. In addition the limb may be artificially blocked in order to measure a regain and post obstruction response of the physiological signs. The PAD screening device measurements are optionally performed at different position/angles of the limb in order to determine the anatomical blockage properties.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

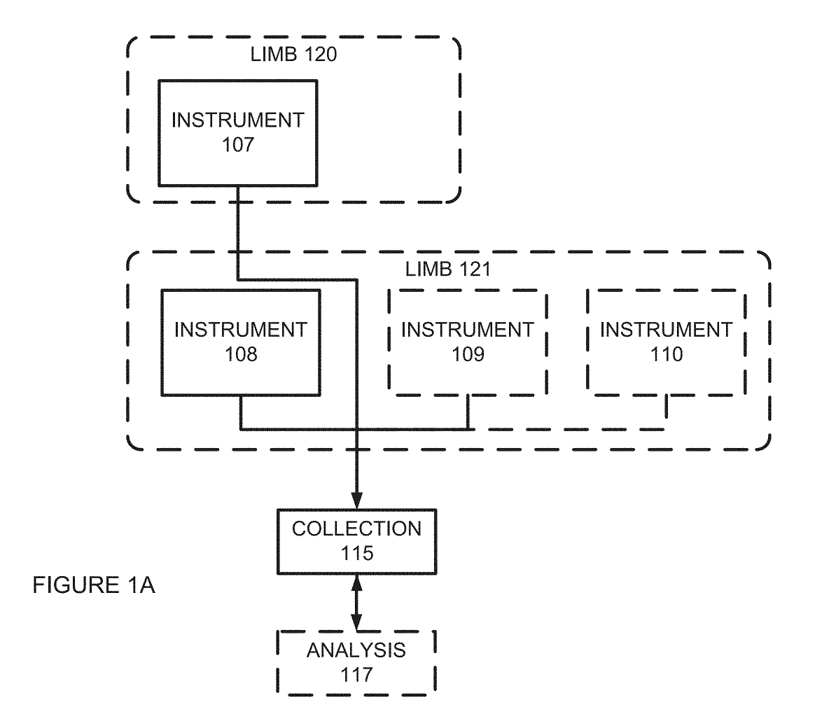

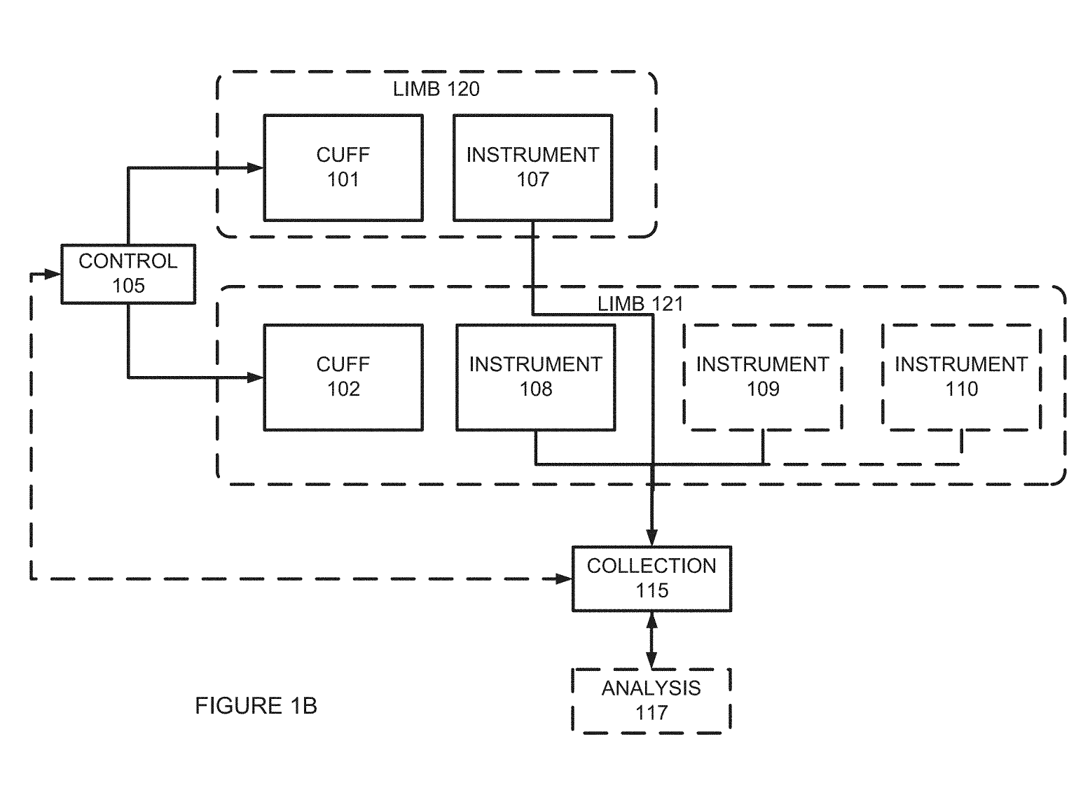

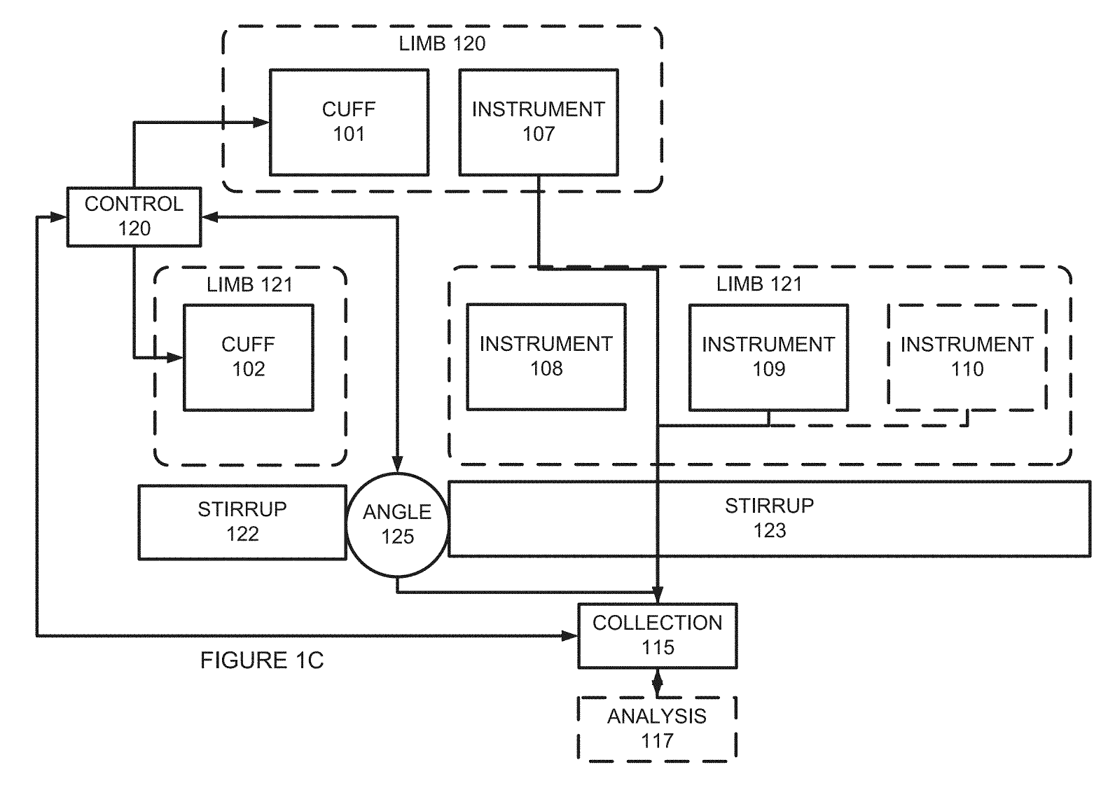

Reference is now made to FIG. 1A, which is a simplified block diagram illustration of an example embodiment of the invention.

FIG. 1A depicts:

a first instrument 107 for sensing blood flow in a first limb 120. In some embodiments the first limb 120 is an arm. In some embodiments the first limb 120 optionally refers to measuring two arms (not shown). In some embodiments the first limb 120 optionally refers to measuring two arms (not shown) simultaneously;

a second instrument 108 for sensing blood flow in a second limb 121. In some embodiments the second limb 121 is a leg. In some embodiments the second limb 121 optionally refers to measuring two legs (not shown). In some embodiments the first limb 121 optionally refers to measuring two legs (not shown) simultaneously;

a signal or data collection unit 115, for collecting data from the first instrument 107 and the second instrument 108; and

optionally an analysis unit 117 for analyzing the signals and/or data collected by the data collection unit 115.

In some embodiments all sensor caps convey data to a central controller which incorporates the data into a PAD detection algorithm.

In some embodiments the central controller optionally integrates changes of signals over time.

In some embodiments the central controller integrates absolute and relative changes of signals over a distance from a site of flow obstruction.

In some embodiments the central controller integrates absolute and relative changes of signals between different limbs.

In some embodiments the central controller optionally produces an overall risk assessment of a likelihood of having a certain degree of arterial flow blockage in specific anatomical parts of a tested limb, described as disease free, mild disease and severe disease.

In some embodiments the central controller optionally provides a simultaneously measured ankle-brachial index (ABI).

In some embodiments, additional instruments 109 110 are optionally placed along the second limb 121, optionally at various locations as described above, such as, by way of some non-limiting examples, behind a knee, at a back of an ankle, and on a toe, and signals produced by the additional instruments 108 110 are optionally collected by the collection unit 115 and analyzed by the analysis unit.

Reference is now made to FIG. 1B, which is a simplified block diagram illustration of an example embodiment of the invention.

FIG. 1B is intended to depict inflatable cuffs for restricting blood flow in the limbs and a controller for controlling the inflatable cuffs and optionally for controlling a collection of data synchronized with inflating the cuffs.

FIG. 1B depicts:

a first inflatable cuff 101 for placing on a first limb 120;

a first instrument 107 for sensing blood flow in the first limb 120;

a second inflatable cuff 102 for placing on a second limb 121;

a second instrument 108 for sensing blood flow in the second limb 121;

a controller for controlling inflation of the inflatable cuffs 101 102;

a signal or data collection unit 115, for collecting data from the first instrument 107 and the second instrument 108, optionally communicating and/or under control of the controller 105; and optionally an analysis unit for analyzing the signals and/or data collected by the data collection unit 115.

In some embodiments, additional instruments 109 110 are optionally placed along the second limb 121, as described above with reference to FIG. 1A.

Reference is now made to FIG. 1C, which is a simplified block diagram illustration of an example embodiment of the invention.

FIG. 1C is intended to depict adjustable stirrups for adjusting an angle between a first, upstream portion of the second limb, such as a thigh, and a second, downstream portion of the second limb, such as a leg, optionally automatic readout of the angle, and optionally automatic adjustment of the angle.

FIG. 1C depicts:

a first inflatable cuff 101 for placing on a first limb 120;

a first instrument 107 for sensing blood flow in the first limb 120;

a second inflatable cuff 102 for placing on a first, upstream portion of a second limb 121;

a first stirrup 122 for supporting a first portion of a second limb, the first portion being an upstream portion of the second limb 121, such as a thigh portion is upstream of a leg portion when described with reference to arterial blood flow;

a second stirrup 123 for supporting a second, downstream portion of the second limb 121, the first portion being an upstream portion of the second limb, such as a thigh portion is upstream of a leg portion when described with reference to arterial blood flow;

a second instrument 108 for sensing blood flow in a second, downstream portion of the second limb 121;

a controller for controlling inflation of the inflatable cuffs 101 102;

a signal or data collection unit 115, for collecting data from the first instrument 107 and the second instrument 108, optionally communicating and/or under control of the controller 105, and optionally collecting an angle readout from an angle reading unit 125 which reads an angle between the first stirrup 122 and the second stirrup 123; and

optionally an analysis unit for analyzing the signals and/or data collected by the data collection unit 115.

In some embodiments, additional instruments 109 110 are optionally placed along the second, downstream portion of the second limb 121.

In some embodiments a reading is provided of an angle in which blood flow of a specific limb is compromised.

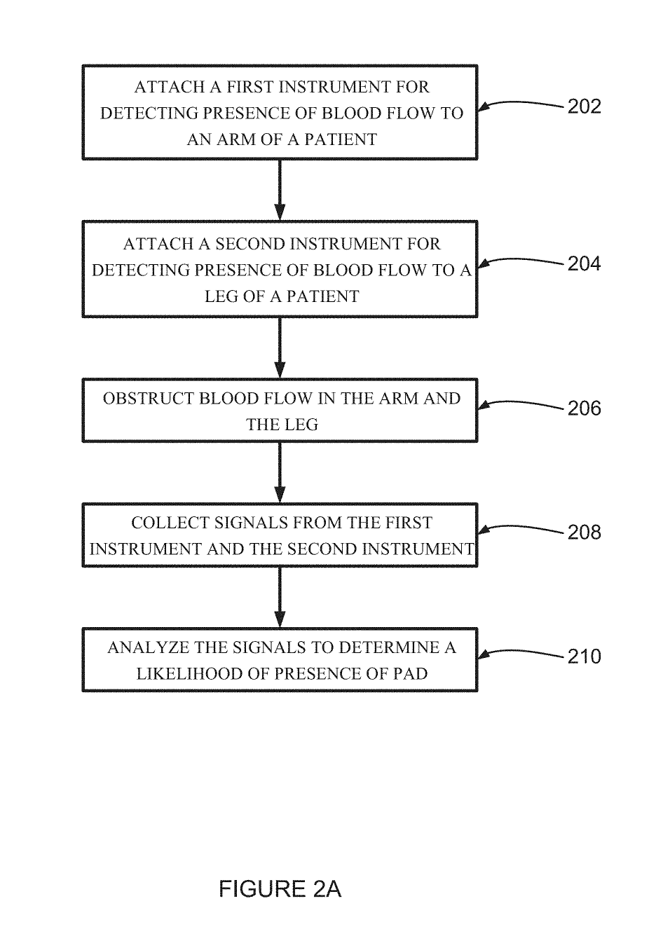

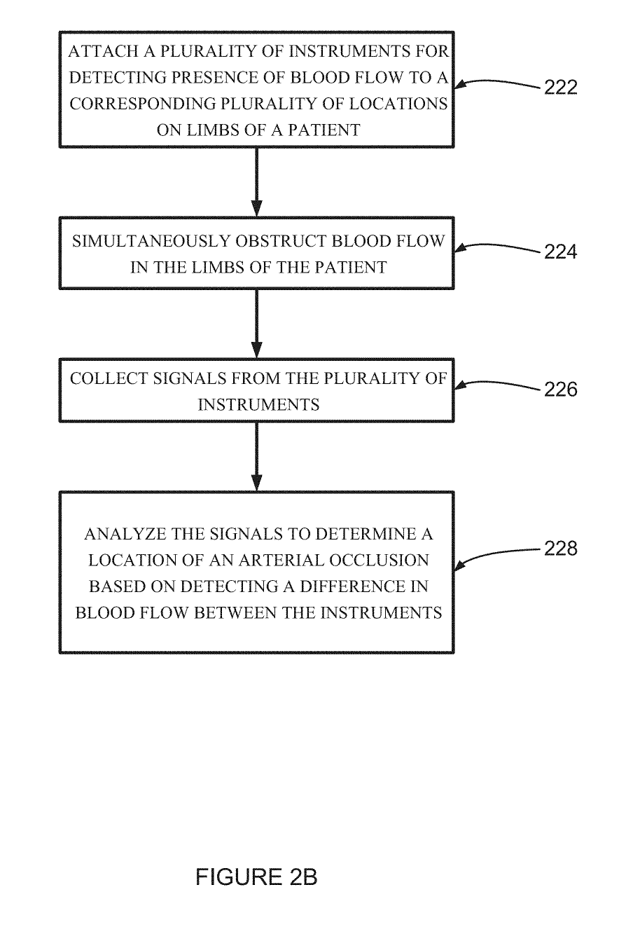

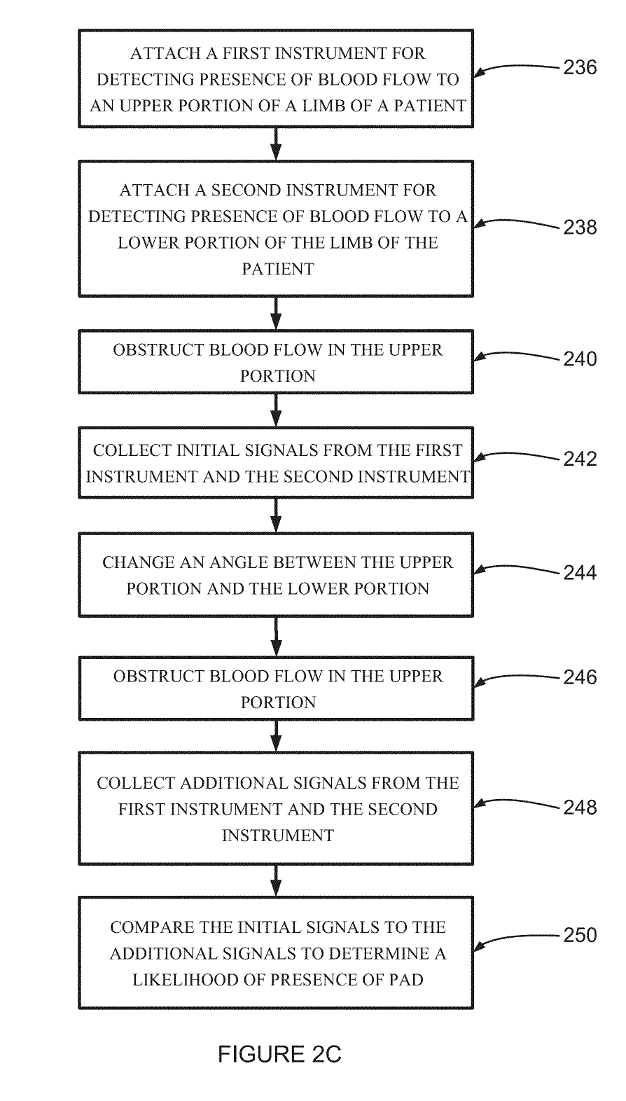

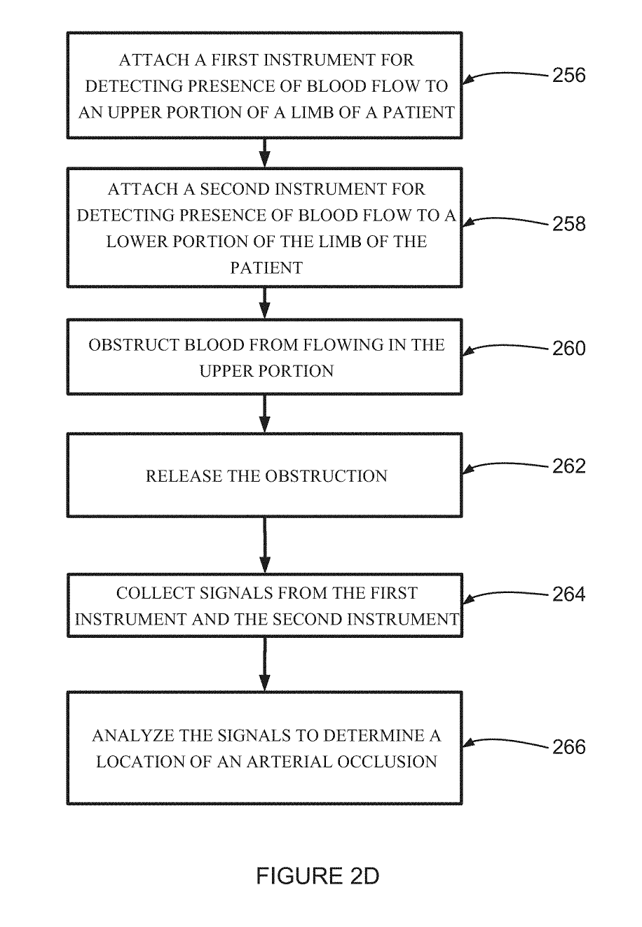

Reference is now made to FIG. 2A, which is a simplified flow chart illustration of an example embodiment of the invention.

The method of FIG. 2A describes a simultaneous measurement of blood flow signals from an arm and a leg, and analyzing the signals, potentially decreasing or eliminating errors caused by non-simultaneous measurements, and potentially making a comparison between arm and leg measurements, such as blood pressure measurements, more accurate and more reflective of likelihood of existence of PAD.

The method of FIG. 2A includes:

attaching a first instrument for detecting presence of blood flow to an arm of a patient (202);

attaching a second instrument for detecting presence of blood flow to a leg of a patient (204);

obstructing blood flow in the arm and the leg (206);

collecting signals from the first instrument and the second instrument (208); and

analyzing the signals to determine a likelihood of presence of PAD (210).

In some embodiments, a leg of a patient is placed in an adjustable stirrup, and an angle between the leg and a thigh of the patient is measured, and optionally used as part of the analyzing the signals to determine a likelihood of presence of PAD.

In some embodiments, a thigh of a patient is placed in an adjustable thigh stirrup and a leg of the patient is placed in an adjustable leg stirrup, and an angle between the leg stirrup and the thigh stirrup is measured, and optionally used as part of the analyzing the signals to determine a likelihood of presence of PAD.

In some embodiments, a motor is used to automatically adjust the angle between the leg stirrup and the thigh stirrup.

In some embodiments, a controller is used to instruct the motor to automatically adjust the angle between the leg stirrup and the thigh stirrup to various different angles, and optionally to instruct a data collection unit to collect signals from at least the first instrument and the second instrument for the different angles.

Reference is now made to FIG. 2B, which is a simplified flow chart illustration of an example embodiment of the invention.

The method of FIG. 2B describes a simultaneous measurement of blood flow signals from several anatomic locations of a patient's limbs, analyzing the signals, and potentially deducing a location of an arterial occlusion.

The method of FIG. 2B includes:

attaching a plurality of instruments for detecting presence of blood flow to a corresponding plurality of locations on limbs of a patient (222);

simultaneously obstructing blood flow in the limbs of the patient (224);

collecting signals from the plurality of instruments (226); and

analyzing the signals to determine a location of an arterial occlusion based, at least in part, on detecting a difference in blood flow between the instruments (228).

In some embodiments absolute and relative changes of signals between positions and angles of the tested limb are optionally taken into account in estimating likelihood of PAD, as mentioned in the Overview section above.

In some embodiments an angle is provided at which blood flow of a specific limb is compromised.

Reference is now made to FIG. 2C, which is a simplified flow chart illustration of an example embodiment of the invention.

The method of FIG. 2C describes a simultaneous measurement of blood flow signals from an upper and a lower portion of a patient's limb, changing an angle between the upper and the lower portion, analyzing the signals at different angles, and potentially deducing a likelihood of presence of PAD.

The method of FIG. 2C includes:

attaching a first instrument for detecting presence of blood flow to an upper portion of a limb of a patient (236);

attaching a second instrument for detecting presence of blood flow to a lower portion of the limb of the patient (238);

obstructing blood flow in the upper portion (240);

collecting initial signals from the first instrument and the second instrument (242);

changing an angle between the upper portion and the lower portion (244);

obstructing blood flow in the upper portion (246);

collecting additional signals from the first instrument and the second instrument (248); and

comparing the initial signals to the additional signals to determine a likelihood of presence of PAD (250).

In some embodiments, the obstructing blood flow in the upper portion includes obstructing the blood flow until signals from the first instrument and the second instrument indicate no blood is flowing, followed by releasing the obstruction.

In some embodiments the changing the angle between the upper portion and the lower portion and the collecting additional signals from the first instrument and the second instrument are repeated a numbers of times.

In some embodiments the changing the angle between the upper portion and the lower portion is incremented by a specific angle such as, by way of some non-limiting examples, 10 degrees, 10 degrees, 30 degrees, 45 degrees, 60 degrees and 80 degrees.

In some embodiments the changing the angle includes changing the angle from a straightest angle to a most-bent angle of the limb.

In some embodiments the changing the angle is stopped when one of the first instrument and the second instrument indicate a drop in physiological parameters affected by blood flow by a specific percentage which correlates with a clinically significant artery occlusion, such as, by way of a non limiting example, 10%, 20%, 30%, 40%, 50%, 60%, 70%, 80%, 90%, 100%, 150% and 200%.

In some embodiments the changing the angle is stopped when one of the first instrument and the second instrument indicate no blood is flowing.

Reference is now made to FIG. 2D, which is a simplified flow chart illustration of an example embodiment of the invention.

The method of FIG. 2D describes a method for determining a location of an arterial occlusion.

The method of FIG. 2D includes:

attaching a first instrument for detecting presence of blood flow to an upper portion of a limb of a patient (256);

attaching a second instrument for detecting presence of blood flow to a lower portion of the limb of the patient (258);

obstructing blood from flowing in the upper portion (260);

releasing the obstruction (262);

collecting signals from the first instrument and the second instrument (264); and

analyzing the signals to determine a location of an arterial occlusion (266).

In some embodiments a third instrument for detecting presence of blood flow is attached to another limb of the patient, blood is also obstructed from flowing in the other limb and the obstructing is released, signals are collected from the third instrument, and the signals are analyzed to determine a location of an arterial occlusion.

In some embodiments determining the location of the arterial occlusion is performed by comparing blood flow in the first instrument and in the second instrument to blood flow in the third instrument.

In some embodiments, when the analyzing determines that the first instrument measured blood flow higher than the second instrument by a specific threshold, the location of the arterial occlusion is determined to be between the first instrument and the second instrument.

In some embodiments, when the analyzing determines that both the first instrument and the second instrument measure a decrease in blood flow relative to the third instrument the location of the arterial occlusion is determined to be upstream of the first instrument.

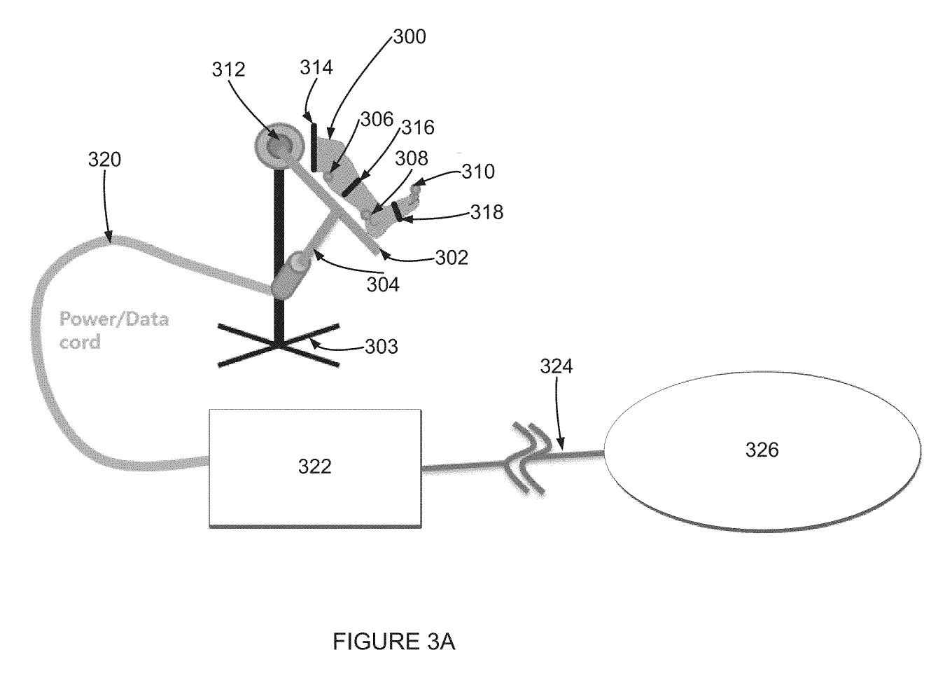

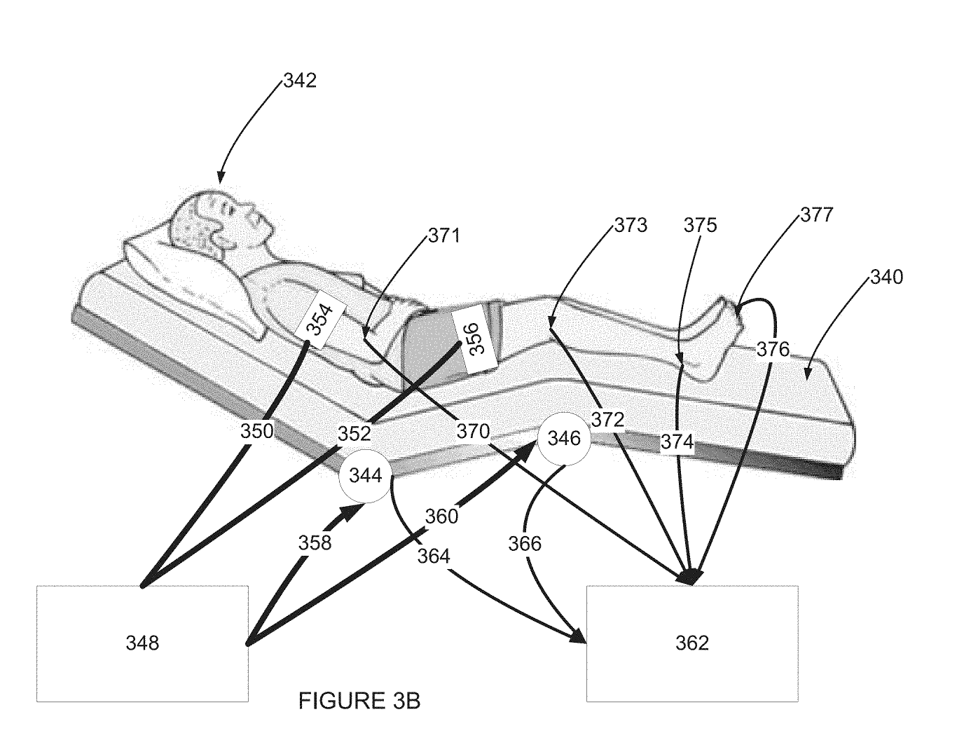

Reference is now made to FIGS. 3A-B, which is are simplified illustrations of an example embodiment of the invention.

FIGS. 3A-B are intended to depict a view of a simple embodiment with a single leg stirrup, a mechanical device to control leg angle, and three sensing locations on the leg.

FIGS. 3A-B depict a stand 303, connected to a piston 304 supporting a leg rest or stirrup 302. The stirrup 302 is connected to the stand 303 by a hinge 312, enabling supporting a leg 300 at various angles.

In some embodiments, the piston 304 is optionally connected to (320) and controlled by a controller 322, optionally a computer, which controls extension of the piston and angle of the stirrup 302.

FIGS. 3A-B depict and example embodiment including three blood flow sensing locations 306 308 310. A first sensor 306 is optionally located at a back of a knee. A second sensor 308 is optionally located at a back of an angle. A third sensor 310 is optionally located at a toe.

FIGS. 3A-B also depict a connection 324 from the computer or controller 322 to various blood flow sensors 326 located at the three locations 306 308 310. The blood flow sensors are optionally one or more of the blood flow sensors described above at each one of the sensing locations 306 308 310.

In some embodiments, signals from the blood flow sensors at the blood flow sensing locations 306 308 310 are optionally compared to a signal from one or more arm blood flow sensor(s) (not shown in FIGS. 3A-B, but shown in FIGS. 1A-C).

When blood flow is detected to be significantly less in all the sensing locations 306 308 310 relative to the arm, a high likelihood of PAD is deduced, and optionally a location of an arterial occlusion is deduced to be at or above the knee.

When blood flow is detected to be significantly less in the ankle sensing location 308 and/or the toe sensing location 310 relative to the knee sensing location 306, a high likelihood of PAD is deduced, and optionally a location of an arterial occlusion is deduced to be below the knee.

When blood flow is detected to be significantly less in the toe sensing location 310 relative to the ankle sensing location 308, a high likelihood of PAD is deduced, and optionally a location of an arterial occlusion is deduced to be between the ankle and the toe.

In some embodiments a multi-sensor PAD screening device contains several multi-sensor heads in order to detect changes in blood flow derived parameters such as auscultation, temperature, and tissue oxygen saturation.

In some embodiments sensor heads are optionally placed in anatomical locations which are known to those skilled in the art as providing clear blood flow derived or associated signals. The locations are such that physiological parameters can easily be measured by the sensor heads. The sensor heads optionally contain an array of sensors that are arranged in a design such that no special training is needed for placing the sensor heads and the sensor head can optionally be placed by any medical team person.

In some embodiments, a sensor array is optionally controlled via a computer or some other device in order to provide significant results using one or more sensors to measure physiological vital signs.

A device for screening for PAD according to an embodiment of the invention optionally provides real time results from several anatomic locations simultaneously in order to enable comparison between different parts of a limb for high confidence whether the limb has a blockage, and if so, where the blockage is.

In some embodiments the device algorithm estimates the blockage level using physiological parameters. The device algorithm optionally uses limb external blockage devices such as pressure cuff or other devices, after which post obstructive flow allows enhancement of the physiological parameters which are optionally measured such as tissue relief saturation and temperature regain.

In some embodiments the device optionally includes a limb lifting device which optionally enables measuring at a continuous range of limb positions and angles in order to detect changes in physiological signs. The lifting device is optionally such that enables determining critical angles/positions. The angles/positions are optionally used to analyze blockage properties.

In some embodiments the device optionally includes several multi-sensor heads. Each head optionally includes an array of sensors. A user, nurse\M.D.\intern\trained personnel, optionally places the sensor heads approximately at designated anatomical locations.

In some embodiments the device optionally examines signals from all sensors in order to determine the best sensors to use. After sensor heads are placed a lifting device optionally changes limb position\angle continuously while recording physiological signs. Optionally, once the signals pass a predetermined criteria the measurement optionally stops. Optionally the data is plotted, including estimation of a blockage level and location.

In some embodiments the device optionally uses an above-the-knee obstruction in order to determine a time constant to regain baseline physiological measurements.

In some embodiments the device optionally uses a relative comparison of measurements between limbs of a patient, and optionally also an absolute examination from a sick and a healthy pool of patients, and optionally calibrates a detection algorithm accordingly, in order determine a level of blockage.

In some embodiments the device optionally uses dynamic measurement including changing limb angle from perpendicular to ground to parallel to ground in order to determine a critical angle where physiological vital signs reduce by a predetermined amount.

In some embodiments the device optionally includes ABI examination as well as other examinations.

In some embodiments the device optionally includes an algorithm in order to have repeatable (non-M.D. dependent) measurements of blood flow derived parameters and of angles while determining the blockage level.

In some embodiments the device is optionally fully automated in order to reduce human error.

In some embodiments the device optionally includes multi-sensor heads including several sensors in each head to increase significance of physiological vital signs.

In some embodiments the device optionally contains multi-sensor heads including several sensors in each head in order to prevent human error when locating the sensor heads.

Potentially, a device for PAD screening according to an example embodiment of the invention has a high reliability both in localization of an arterial occlusion and in estimating a blockage degree.

Potentially, a device for PAD screening according to an example embodiment of the invention “takes the human out of the process”, reducing human error both in operating the device and with a result analysis which produces repetitive results.

Reference is now made to FIG. 3B, which is a simplified illustration of an example embodiment of the invention.

FIG. 3B depicts a system which supports a patient 342 at various torso to thigh to leg angles, optionally controls the angles, optionally performs blood flow obstruction and/or relief under automated control, optionally measures angles and/or blood flow derived physiological parameters, and optionally analyzes data collected from the system and optionally patient-related anthropometric and medical data.

FIG. 3B does not depict separate support for two thighs and two legs, however, some embodiments of the invention do include separate support, control of angles, and measurements for the two thighs and the two legs.

FIG. 3B depicts an adjustable patient support component 340, with a controller 348 for optionally controlling one or more of:

controlling 358 a torso to thigh angle 344;

controlling 360 a thigh to leg angle 346;

controlling 350 an inflatable/deflatable arm cuff 354 for restricting/releasing arm blood flow; and

controlling 352 an inflatable/deflatable thigh cuff 356 for restricting/releasing leg blood flow.

FIG. 3B also depicts a signal collection and analysis unit 362 for:

optionally reading 364 a torso-to-thigh angle 344;

optionally reading 366 a thigh-to-leg angle 346;

optionally reading 370 blood flow derived physiological parameters from one or more sensors attached to a location 371 on the patient's arm;

optionally reading 372 blood flow derived physiological parameters from one or more sensors attached to a location 373 behind the patient's knee;

optionally reading 374 blood flow derived physiological parameters from one or more sensors attached to a location 375 behind the patient's knee; and

optionally reading 376 blood flow derived physiological parameters from one or more sensors attached to a location 377 at the patient's toe(s).

In some embodiments the signal collection and analysis unit 362 optionally receives signals from multiple sensors per location, and optionally analyzes the signals, optionally filtering out and discarding noisy or unintelligible signals.

In some embodiments the signal collection and analysis unit 362 optionally analyzes the signals received, and optionally additional data such as, by way of a non-limiting example, patient-related anthropometric and medical data, and optionally a determination of a likelihood of presence of PAD and/or a location of a determined blood flow occlusion.

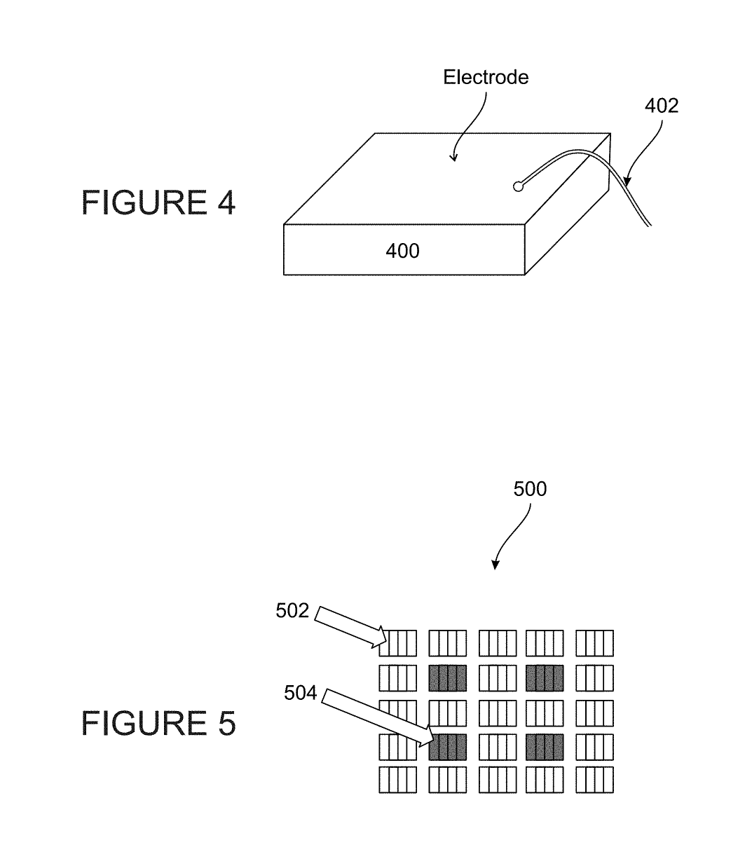

Reference is now made to FIG. 4, which is a simplified illustration of a piezoelectric crystal for use as a piezoelectric sensor according to an example embodiment of the invention.

A piezoelectric crystal produces voltage across opposite faces if the crystal in response to pressure. The piezoelectric crystal is optionally used as a blood flow or pulse sensor by sensing pulse when attached to a sensing location on a patient.

FIG. 4 depicts a piezoelectric crystal 400, with a wire 402 attached to one side of the piezoelectric crystal 400. In some embodiments a second wire (not shown) is attached to an opposite side of the piezoelectric crystal 400. In some embodiments the opposite side of the piezoelectric crystal 400 is attached to a conducting surface serving as a container for the piezoelectric crystal 400.

Reference is now made to FIG. 5, which is a simplified illustration of an array of sensors according to an example embodiment of the invention.

FIG. 5 is intended to show how an array of sensors covers a larger area than a single sensor, and can thus potentially compensate for shifts in placement of the sensors.

FIG. 5 depicts an enhanced reflection pulse oxymetry sensor including an array of light emitters 504 and an array of light detectors 502.

In some embodiment a single light emitter such as the light emitters in the array of light emitters 504, coupled with a single light detector such as the light detectors in the array of light detectors 502, is sufficient to provide light for the pulse oxymetry sensor.

In some embodiment a single light emitter such as the light emitters in the array of light emitters 504, coupled with an array of light detectors 502, is sufficient to operate the pulse oxymetry sensor, and even cover a larger area than a single light detector.

In some embodiment an array of light emitters 504, coupled with an array of light detectors 502, operates the pulse oxymetry sensor.

In some embodiment several arrays of light emitters 504, coupled with several arrays of light detectors 502, as depicted in FIG. 5, operates the pulse oxymetry sensor, and covers a larger area than the embodiments described above.

In some embodiments, where multi-sensor caps include more than one sensor, a signal analyzer optionally evaluates signal quality for each sensor and optionally selects one or more sensors which provide good quality signals for measurement at a specific location.

It is expected that during the life of a patent maturing from this application many relevant devices for sensing pulse and/or blood flow will be developed and the scope of the terms pulse sensor and/or blood flow sensor are intended to include all such new technologies a priori.

As used herein the term “about” refers to ±10%.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for assessing limb circulation and Peripheral Artery Disease (PAD), comprising:
a plurality of instruments for measuring blood flow derived physiological parameters in response to positional maneuvers of the limbs;
a data collection unit attached to each one of the instruments for receiving signals from each one of the instruments; and
a data analysis unit for determining a likelihood of presence of PAD based, at least in part, on the received signals.

2-3. (canceled)

4. The system of claim 1 and further comprising:
a plurality of inflatable and deflatable cuffs for blocking circulation of a limb;
an electric pump for inflating the inflatable cuffs; and
an inflation controller for controlling inflation and release of pressure of the inflatable cuffs arranged to maintain same pressure at each one of the plurality of inflatable cuffs.

5-6. (canceled)

7. The system of claim 1 and further comprising an adjustable thigh stirrup for supporting a thigh of a patient and an adjustable leg stirrup for supporting a leg of a patient, functionally attached to a motor for adjusting at least one angle selected from a group consisting of:
between the thigh of the patient and the leg of the patient; and
between the thigh of the patient and torso of the patient.

8. (canceled)

9. The system of claim 7 and further comprising an angle measurement device for measuring at least one of the angles and transmitting the angle measurement.

10. The system of claim 1 and further comprising at least one sleeve selected from a group consisting of:
a sleeve for placing at least one of the plurality of the instruments at a location behind a knee of a patient;
a sleeve for placing at least one of the plurality of the instruments at a medial aspect of an ankle of a patient;
a sleeve for placing at least one of the plurality of the instruments at a toe of a patient;
a sleeve for placing at least one of the plurality of the instruments in a vicinity of a dorsalis pedis artery; and
a sleeve for placing at least one of the plurality of the instruments at an inner elbow of a patient.

11. (canceled)

12. A method for determining a likelihood of presence of Peripheral Artery Disease (PAD), comprising:
attaching a first instrument for detecting presence of blood flow derived physiological parameters to an arm of a patient;
attaching a second instrument for detecting presence of blood flow derived physiological parameters to a leg of a patient;
obstructing blood flow in the arm and the leg followed by relieving the obstruction;
collecting signals from the first instrument and the second instrument;
analyzing the signals to determine a likelihood of presence of PAD,
changing a limb angle;
collecting signals from the first instrument and the second instrument at a changed limb angle; and
analyzing the signals to determine a likelihood of presence of PAD, based, at least in part, on comparing the signals at different angles,
thereby determining a likelihood of presence of PAD.

13-15. (canceled)

16. The method of claim 12 in which the second instrument is attached behind a knee of the patient.

17. (canceled)

18. The method of claim 12 and further comprising determining a location of an arterial occlusion or a partial arterial occlusion based, at least in part, on the analyzing the signals.

19-20. (canceled)

21. The method of claim 12 in which the obstructing blood flow comprises simultaneously inflating an arm cuff and a thigh cuff.

22. The method of claim 12 in which the obstructing blood flow comprises maintaining a same pressure at each one of a plurality of inflatable cuffs.

23. (canceled)

24. The method of claim 12 and further comprising placing a thigh of a patient in an adjustable thigh stirrup and placing a leg of the patient in an adjustable leg stirrup and measuring an angle between the leg stirrup and the thigh stirrup,
and further comprising using a motor to automatically adjust at least one angle selected from a group consisting of:
between the leg stirrup and the thigh stirrup; and
between the thigh of the patient and torso of the patient.

25-27. (canceled)

28. The method of claim 12 in which a plurality of instruments are attached to a leg of a patient for detecting presence and characteristics of blood flow derived physiological parameters and further comprising determining a location of an arterial occlusion based, at least in part, on detecting a difference in blood flow derived parameters between the plurality of instruments.

29. (canceled)

30. The method of claim 28 and further comprising determining a location of a partial arterial occlusion based, at least in part, on detecting a difference in blood flow derived parameters between the plurality of instruments.

31-34. (canceled)

35. A method for determining a likelihood of presence of Peripheral Artery Disease (PAD), comprising:
attaching a first instrument for detecting presence of blood flow to a proximal portion of a limb of a patient;
attaching a second instrument for detecting presence of blood flow to a distal portion of the limb of the patient;
obstructing blood flow in the upper portion;
collecting initial signals from the first instrument and the second instrument;
changing an angle between the proximal portion and the distal portion;
obstructing blood flow in the upper portion;
collecting additional signals from the first instrument and the second instrument; and
comparing the initial signals to the additional signals to determine a likelihood of presence of PAD,
thereby determining a likelihood of presence of PAD.

36-41. (canceled)

42. The method of claim 35 in which the changing the angle between the proximal portion and the distal portion comprises continuous changing of the angle between two predetermined angles.

43. The method of claim 42 in which the changing the angle between the proximal portion and the distal portion comprises stopping the changing the angle when one of the first instrument and the second instrument indicate a drop in blood flow by a specific percentage.

44. The method of claim 42 in which the changing the angle between the upper portion and the lower portion comprises stopping the changing the angle when one of the first instrument and the second instrument indicate no blood is flowing.

45. (canceled)

46. The method of claim 35 and further comprising:
attaching a third instrument for detecting presence of blood flow to another limb of the patient;
obstructing blood from flowing in the other limb;
releasing the obstruction in the other limb;
collecting signals from the third instrument; and
analyzing the signals to determine a location of an arterial occlusion,
in which the determining the location of the arterial occlusion is performed by comparing blood flow in the first instrument and in the second instrument to blood flow in the third instrument.

47. (canceled)

48. The method of claim 35 in which when the comparing determines that the first instrument measured blood flow higher than the second instrument by a specific threshold, the location of the arterial occlusion is determined to be between the first instrument and the second instrument.

49. The method of claim 46 in which when the analyzing determines that both the first instrument and the second instrument measure a decrease in blood flow relative to the third instrument the location of the arterial occlusion is determined to be upstream of the first instrument.
